(19) United States
(12) Patent Application Publication
(10) Pub. No.: US 2022/0261520 A1
(43) Pub. Date: Aug. 18, 2022

(54) QUALITY PREDICTION MODEL GENERATION METHOD, QUALITY PREDICTION MODEL, QUALITY PREDICTION METHOD, METAL MATERIAL MANUFACTURING METHOD, QUALITY PREDICTION MODEL GENERATION DEVICE, AND QUALITY PREDICTION DEVICE

(71) Applicant: JFE Steel Corporation, Tokyo (JP)

(72) Inventor: Hiroyasu Shigemori, Chiyoda-ku, Tokyo (JP)

(73) Assignee: JFE Steel Corporation, Tokyo (JP)

(21) Appl. No.: 17/621,801

(22) PCT Filed: Jun. 9, 2020
(86) PCT No.: PCT/JP2020/022637

§ 371 Date: Dec. 22, 2021

(30) Foreign Application Priority Data
Jul. 22, 2019 (JP) 2019-134701

(51) Int. Cl. G06F 30/27

(52) U.S. Cl. CPC G06F 30/27

(57) ABSTRACT

A quality prediction model generation method for a metal material manufactured through one or more processes includes: a first collection step of collecting a manufacturing condition of each of the processes for each of predetermined areas of the metal material; a second collection step of evaluating and collecting quality of the metal material manufactured through each process for each of the predetermined areas; a storage step of storing the manufacturing condition of each process and the quality of the metal material manufactured under the manufacturing condition in association with each other for each of the predetermined areas; and a model generation step of generating a quality prediction model that predicts quality of the metal material for each of the predetermined areas based on the stored manufacturing condition for each of the predetermined areas in each process.

### CROSS REFERENCE TO RELATED APPLICATIONS

This is the U.S. National Phase application of PCT/JP2020/022637, filed Jun. 9, 2020, which claims priority to Japanese Patent Application No. 2019-134701, filed Jul. 22, 2019, the disclosures of these applications being incorporated herein by reference in their entireties for all purposes.

### FIELD OF THE INVENTION

The present invention relates to a quality prediction model generation method, a quality prediction model, a quality prediction method, a metal material manufacturing method, a quality prediction model generation device, and a quality prediction device.

### BACKGROUND OF THE INVENTION

For methods of predicting quality with respect to certain requirement conditions, for example, there is a known method of calculating the distances between a plurality of past observed conditions stored in a record database and a desired requirement condition, calculating the weights of observed data (record data) based on the calculated distances, creating a function for fitting the neighborhood of the requirement condition based on the calculated weights, and predicting quality with respect to the requirement condition using the created function (refer to Patent Literature 1 to 8, for example).

### PATENT LITERATURE

Patent Literature 1: Japanese Patent Application Laid-open No. 2004-355189
Patent Literature 2: Japanese Patent Application Laid-open No. 2006-309709
Patent Literature 3: Japanese Patent Application Laid-open No. 2008-112288
Patent Literature 4: Japanese Patent Application Laid-open No. 2009-230412
Patent Literature 5: Japanese Patent Application Laid-open No. 2014-013560
Patent Literature 6: Japanese Patent Application Laid-open No. 2014-071858
Patent Literature 7: Japanese Patent Application Laid-open No. 2014-071859
Patent Literature 8: Japanese Patent Application Laid-open No. 2017-120638

### SUMMARY OF THE INVENTION

In the methods disclosed in Patent Literature 1 to 8, quality with respect to a certain requirement condition is calculated from data stored in a record database. This record database stores therein the record values of a plurality of manufacturing conditions and the record values of the quality of a metal material manufactured under these manufacturing conditions. As each of these manufacturing conditions and quality for one metal material, one representative value such as an average value is stored.

On the other hand, record values of manufacturing conditions for each process are collected in detail in the longitudinal direction of the metal material by sensors. However, in conventional techniques, the record values of manufacturing conditions collected in such detail have not been effectively utilized, which limits the improvement in prediction accuracy of the quality of metal materials.

Aspects of the present invention have been made in consideration of the above, and it is an object according to aspects of the present invention to provide a quality prediction model generation method, a quality prediction model, a quality prediction method, a metal material manufacturing method, a quality prediction model generation device, and a quality prediction device capable of predicting quality with respect to a certain manufacturing condition with high accuracy.

In order to solve the above-described problem and achieve the object, a quality prediction model generation method according to aspects of the present invention for a metal material manufactured through one or more processes includes: a first collection step of collecting a manufacturing condition of each of the processes for each of predetermined areas of the metal material; a second collection step of evaluating and collecting quality of the metal material manufactured through each process for each of the predetermined areas; a storage step of storing the manufacturing condition of each process and the quality of the metal material manufactured under the manufacturing condition in association with each other for each of the predetermined areas; and a model generation step of generating a quality prediction model that predicts quality of the metal material for each of the predetermined areas based on the stored manufacturing condition for each of the predetermined areas in each process.

Moreover, in the above-described quality prediction model generation method according to aspects of the present invention, each of the predetermined areas is determined based on a travel distance of the metal material in a conveyance direction in each process.

Moreover, in the above-described quality prediction model generation method according to aspects of the present invention, before the storage step, a third collection step collects at least one or more of whether a leading end and a trailing end of the metal material have been interchanged in each process, whether a front face and a back face of the metal material have been interchanged in each process, and a cutting position of the metal material in each process, and the storage step identifies the predetermined areas by taking into account, on the metal material in each process, at least one or more of whether the leading end and the trailing end have been interchanged, whether the front face and the back face have been interchanged, and the cutting position, and stores the manufacturing condition of each process and the quality of the metal material produced under the manufacturing condition in association with each other for each of the predetermined areas.

Moreover, in the above-described quality prediction model generation method according to aspects of the present invention, in a case where a shape of the metal material is deformed by going through each process, the storage step identifies the predetermined area by evaluating a volume of the metal material from the leading end, and stores the manufacturing condition of each process and the quality of the metal material manufactured under the manufacturing condition in association with each other for each of the predetermined areas.

Moreover, in the above-described quality prediction model generation method according to aspects of the present invention, the model generation step generates the quality prediction model using machine learning including linear regression, local regression, principal component regression, PLS regression, a neural network, a regression tree, a random forest, and XGBoost.

In order to solve the above-described problem and achieve the object, a quality prediction model according to aspects of the present invention is generated by the quality prediction model generation method.

In order to solve the above-described problem and achieve the object, a quality prediction method according to aspects of the present invention includes predicting quality of a metal material manufactured under a certain manufacturing condition for each of the predetermined areas using a quality prediction model generated by the quality prediction model generation method.

In order to solve the above-described problem and achieve the object, a metal material manufacturing method according to aspects of the present invention includes: fixing a manufacturing condition confirmed during manufacturing; predicting quality of a metal material manufactured under the fixed manufacturing condition for each of predetermined areas by the quality prediction method; and changing the manufacturing condition of a subsequent process based on a predicted result.

Moreover, in the above-described metal material manufacturing method according to aspects of the present invention, the changing the manufacturing condition is performed such that the quality of the manufactured material in every predetermined area included over an entire length of the metal material is within a predetermined control range.

In order to solve the above-described problem and achieve the object, a quality prediction model generation device for a metal material manufactured through one or more processes according to aspects of the present invention includes: a collecting unit configured to collect a manufacturing condition of each of the processes for each of predetermined areas of the metal material; an evaluating unit configured to evaluate and collect quality of the metal material manufactured through each process for each of the predetermined areas; a storing unit configured to store the manufacturing condition of each process and the quality of the metal material manufactured under the manufacturing condition in association with each other for each of the predetermined areas; and a generating unit configured to generate a quality prediction model that predicts quality of the metal material for each of the predetermined areas based on the stored manufacturing condition for each of the predetermined areas in each process.

In order to solve the above-described problem and achieve the object, a quality prediction device according to aspects of the present invention is configured to predict quality of a metal material manufactured under a certain manufacturing condition for each of the predetermined areas using a quality prediction model generated by the quality prediction model generation device.

According to aspects of the present invention, by generating a quality prediction model that associates a manufacturing condition of each process with the quality of a metal material manufactured under the manufacturing condition for each of predetermined areas, the quality of the metal material with respect to a certain manufacturing condition can be predicted more accurately than it is conventionally.

### BRIEF DESCRIPTION OF THE DRAWINGS

FIG. 1 is a block diagram illustrating a configuration of a quality prediction model generation device and a quality prediction device according to an embodiment of the present invention.

FIG. 2 is a flowchart illustrating a flow of a quality prediction model generation method and a quality prediction method according to the embodiment of the present invention.

FIG. 3 is a chart illustrating an example of record data collected by a manufacturing record collection section 11 in the quality prediction model generation method according to the embodiment of the present invention.

FIG. 4 is a chart illustrating an example of record data edited by a manufacturing record edition section 12 in the quality prediction model generation method according to the embodiment of the present invention.

FIG. 5 is a diagram illustrating an example case in which metal materials are manufactured through a plurality of processes in the quality prediction model generation method according to the embodiment of the present invention.

FIG. 6 is a diagram illustrating an example of metal materials in each process in the quality prediction model generation method according to the embodiment of the present invention.

FIG. 7 is a chart illustrating an example of record data edited by an integrated process record edition section in the quality prediction model generation method according to the embodiment of the present invention.

FIG. 8 is a diagram schematically illustrating structures of a conventional record database and a record database according to aspects of the present invention.

FIG. 9 includes charts illustrating prediction errors of a conventional method and a method according to aspects of the present invention in predicting the tensile strength of a highly workable high-strength cold-rolled steel sheet.

FIG. 10 includes charts illustrating prediction errors of the conventional method and the method according to aspects of the present invention in predicting the front and back surface hardness of a thick steel sheet.

FIG. 11 includes charts illustrating error ratios of the conventional method and the method according to aspects of the present invention in predicting the front and back surface defects of a hot-dip galvanized steel sheet.

### DETAILED DESCRIPTION OF EMBODIMENTS OF THE INVENTION

A quality prediction model generation method, a quality prediction model, a quality prediction method, a metal material manufacturing method, a quality prediction model generation device, and a quality prediction device according to an embodiment of the present invention will be described with reference to the drawings.

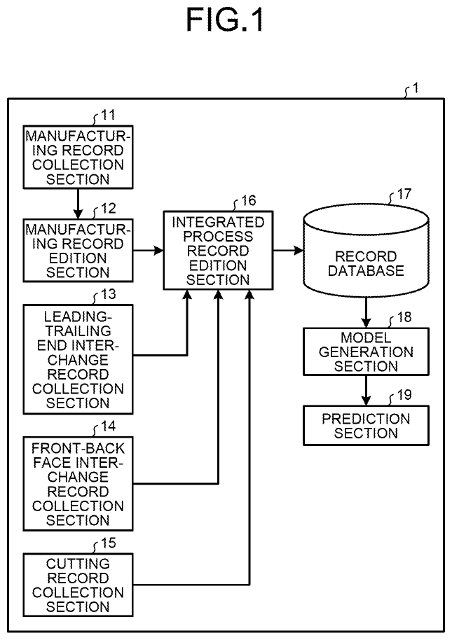

A configuration of the quality prediction device and the quality prediction model generation device according to the present embodiment will be explained with reference to FIG. 1. The quality prediction device is a device for predicting the quality of a metal material that is manufactured through one or more processes. Examples of a metal material in the present embodiment include a steel product, such as a semi-finished product such as a slab, and a product such as a steel sheet manufactured by rolling the slab.

A quality prediction device 1 is, specifically, implemented by a general-purpose information processing device such as a personal computer or a workstation, and includes main components such as a processor consisting of, for example, a central processing unit (CPU), and a memory (main memory) consisting of a random access memory (RAM), a read only memory (ROM), and the like.

As illustrated in FIG. 1, the quality prediction device 1 includes the manufacturing record collection section 11, the manufacturing record edition section 12, a leading-trailing end interchange record collection section 13, a front-back face interchange record collection section 14, a cutting record collection section 15, an integrated process record edition section 16, a record database 17, a model generation section 18, and a prediction section 19. Note that the quality prediction model generation device according to the present embodiment includes the elements of the quality prediction device 1 excluding the prediction section 19. The following also describes the quality prediction model generation device in the description of the quality prediction device 1.

A sensor, not illustrated in the drawings, is connected to the manufacturing record collection section 11. The manufacturing record collection section 11 collects a manufacturing record of each process according to the measurement cycle of the sensor, and outputs the record to the integrated process record edition section 16. The aforementioned “manufacturing record” includes a manufacturing condition of each process and the quality of a metal material manufactured through each process. Examples of the aforementioned “manufacturing condition” include the composition, temperature, pressure, plate thickness, and threading speed of the metal material in each process. In addition, examples of the aforementioned “quality of metal material” include tensile strength and defect contamination rate (the number of defects appearing per unit length).

A manufacturing condition of each process collected by the manufacturing record collection section 11 includes not only an actual measured value of the manufacturing condition measured by the sensor, but also a preset value of the manufacturing condition. In other words, no sensor may be installed in some processes. In such cases, a set value is collected as a manufacturing record instead of a record value.

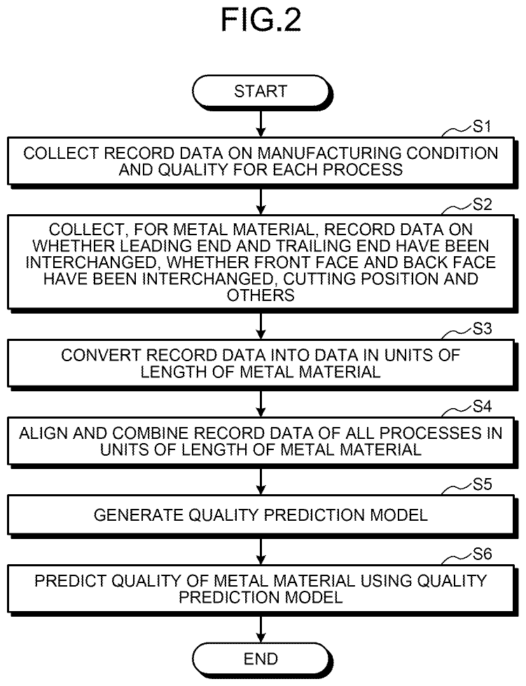

The manufacturing record collection section 11 collects a manufacturing condition of each process for each of predetermined areas of the metal material. In addition, the manufacturing record collection section 11 evaluates and collects the quality of the metal material manufactured through each process for each of the predetermined areas described above. The “predetermined area” mentioned above refers to a certain area in the longitudinal direction of a metal material when the metal material is a slab or steel sheet, for example. This predetermined area is determined based on the travel distance (threading speed) of the metal material based on the conveyance direction in each process. Specific processing by the manufacturing record collection section 11 will be described later (refer to FIG. 2).

In the configuration illustrated in FIG. 1, only one manufacturing record collection section 11 is provided, and the premise is that the data on the manufacturing record of each process (hereinafter referred to as “record data”) is collected by this one manufacturing record collection section 11. For example, however, a plurality of manufacturing record collection sections 11 may be provided to match the number of processes, and the record data of the processes may be collected by the respective manufacturing record collection sections 11.

The manufacturing record edition section 12 edits the record data of each process input from the manufacturing record collection section 11. In other words, the manufacturing record edition section 12 edits the record data collected in units of time by the manufacturing record collection section 11 into record data in units of the length of the metal material, and outputs the data to the integrated process record edition section 16. Specific processing by the manufacturing record edition section 12 will be described later (refer to FIG. 2).

A material loading machine for loading the metal material in each process, which is not illustrated in the drawings, is connected to the leading-trailing end interchange record collection section 13. Through the material loading machine, the leading-trailing end interchange record collection section 13 collects record data on whether the leading end and the trailing end of the metal material have been interchanged (reversed) when the metal material from a previous process is loaded for a subsequent process. Then, the leading-trailing end interchange record collection section 13 outputs, to the integrated process record edition section 16, the record data on whether the leading end and the trailing end of the metal material have been interchanged.

The material loading machine described above is connected to the front-back face interchange record collection section 14. Through the material loading machine, the front-back face interchange record collection section 14 collects record data on whether the front face and the back face of the metal material have been interchanged (reversed) when the metal material from a previous process is loaded for a subsequent process. Then, the front-back face interchange record collection section 14 outputs, to the integrated process record edition section 16, the record data on whether the front face and the back face of the metal material have been interchanged.

A cutting machine for cutting the leading end portion and the trailing end portion of the metal material, which is not illustrated in the drawings, is connected to the cutting record collection section 15. Through the cutting machine, the cutting record collection section 15 collects record data such as a cutting position (distance from the leading end of the metal material at the time of cutting) and the number of cuts (hereinafter referred to as “cutting position and others”) for each metal material. The cutting record collection section 15 then outputs the record data on the cutting position and others of the metal material to the integrated process record edition section 16.

As with the aforementioned manufacturing record collection section 11, only one of each of the leading-trailing end interchange record collection section 13, the front-back face interchange record collection section 14, and the cutting record collection section 15 may be provided, or a plurality of each of these sections may be provided to match the number of processes.

The integrated process record edition section 16 edits the record data input from the manufacturing record edition section 12, the leading-trailing end interchange record collection section 13, the front-back face interchange record collection section 14, and the cutting record collection section 15. The integrated process record edition section 16 stores, in the record database 17, the manufacturing condition of each process and the quality of the metal material manufactured under this manufacturing condition in association with each other for each of the predetermined areas.

The integrated process record edition section 16 identifies a predetermined area while taking into account, for every metal material in each process, whether the leading end and the trailing end have been interchanged, whether the front face and the back face have been interchanged, and the cutting position. The integrated process record edition section 16 then stores, in the record database 17, the manufacturing condition of each process and the quality of the metal material manufactured under this manufacturing condition in association with each other for each of the predetermined areas in a form that is able to distinguish, for the material in each process, whether the leading end and the trailing end have been interchanged, whether the front face and the back face have been interchanged, and the cutting position.

Furthermore, when each process is a rolling process, for example, and the shape of a metal material is deformed as it goes through each process, the integrated process record edition section 16 identifies a predetermined area by evaluating the volume of the metal material from the leading end, and stores, in the record database 17, the manufacturing condition of each process and the quality of the metal material manufactured under this manufacturing condition in association with each other for each of the predetermined areas. This record database 17 stores therein the record data edited by the integrated process record edition section 16.

The model generation section 18 generates a quality prediction model that predicts the quality of a metal material for each of predetermined areas based on the manufacturing condition for each of the predetermined areas in each process stored in the record database 17. The model generation section 18 uses, for example, XGBoost as a machine learning method. Various other methods of machine learning can be used, such as linear regression, local regression, principal component regression, PLS regression, a neural network, a regression tree, and a random forest.

The prediction section 19 uses the quality prediction model generated by the model generation section 18 to predict the quality of a metal material manufactured under a certain manufacturing condition for each of predetermined areas. For example, when the metal material to be predicted is a slab, while conventional methods predict the quality of the entire slab, the present embodiment can predict the quality of the slab for each of predetermined areas in the length direction.

A quality prediction method and a quality prediction model generation method according to the present embodiment will be explained with reference to FIG. 2 to FIG. 7. The quality prediction method according to the present embodiment performs the processing of step S1 to step S6 illustrated in FIG. 2. The quality prediction model generation method according to the present embodiment performs the processing of step S1 to step S5 excluding step S6 illustrated in FIG. 2.

First, the manufacturing record collection section 11 collects record data on a manufacturing condition and quality for each process (step S1). The manufacturing record collection section 11 collects the record data on the manufacturing condition and quality for each metal material and for each process.

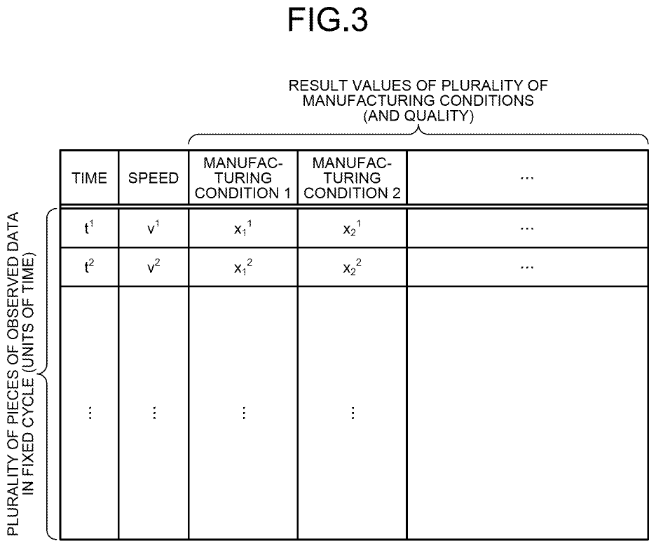

The record data collected by the manufacturing record collection section 11 is, for example, data in which the record values (or set values) of a plurality of manufacturing conditions are arranged by time, as illustrated in the table in FIG. 3. The record data illustrated in FIG. 3 includes times t¹, t² . . . , the speeds of a metal material (threading speed) v¹, v² . . . , and a plurality of manufacturing conditions x₁¹, x₁² . . . , x₂¹, x₂² . . . measured by sensors at the respective times. The record data collected in the final process of a plurality of processes includes an item related to the quality of the metal material in addition to the items illustrated in the FIG. 3.

Then, the leading-trailing end interchange record collection section 13, the front-back face interchange record collection section 14, and the cutting record collection section 15 collect record data on whether the leading end and the trailing end of the metal material have been interchanged in each process, whether the front face and the back face of the metal material have been interchanged in each process, and the cutting position and others of the metal material in each process (step S2).

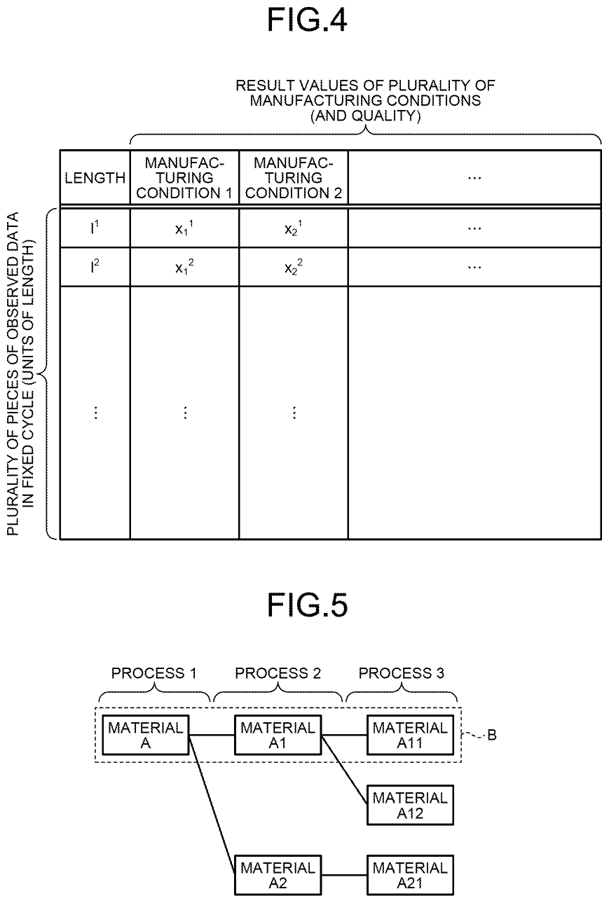

The manufacturing record edition section 12 then converts the record data collected by the manufacturing record collection section 11 into data in units of the length of the metal material (step S3). In other words, the manufacturing record edition section 12 converts the record data collected in units of time, as illustrated in FIG. 3, into record data in units of the length of the metal material, as illustrated in FIG. 4. The following describes a method of converting the record data in FIG. 3 into the record data in FIG. 4.

First, the manufacturing record edition section 12 calculates the position of the metal material at each time in FIG. 3, using the principle that multiplying time and speed (threading speed) records in distance. Next, the manufacturing record edition section 12 detects the leading end and the trailing end of the metal material using a function that record data is recorded when the metal material is passing by the sensors installed for each process, and missing values are recorded when the metal material is not passing. Next, the manufacturing record edition section 12 creates record data corresponding to positions of the metal material from the leading end to the trailing end, except for the time when the metal material is not passing by the sensor.

The data, as it is, is in units of the length of the metal material, but not in a fixed cycle. Thus, the data is converted into record data in units of the length of the metal material and in a fixed cycle by, for example, linear interpolation. In other words, in each process, when the threading speed of the metal material is slow, the record data that can be collected becomes finer, and when the threading speed of the metal material is fast, the record data that can be collected becomes coarser. Therefore, interpolation as described above is used to align the granularity of the record data. The manufacturing record edition section 12 performs the above processing to create the record data in units of the length of the metal material as illustrated in FIG. 4.

Then, the integrated process record edition section 16 combines the record data of all processes, aligning them in units of the length of the metal material (step S4). The integrated process record edition section 16 combines the record data of the manufacturing conditions and the quality of the metal material in all the processes while aligning them in units of the length of the metal material at the output side of the final process based on the record data in units of the length of the metal material created by the manufacturing record edition section 12, and the record data on whether the leading end and the trailing end of the metal material have been interchanged, whether the front face and the back face of the metal material have been interchanged, and the cutting position and others of the metal material collected by the leading-trailing end interchange record collection section 13, the front-back face interchange record collection section 14, and the cutting record collection section 15, respectively.

In this way, the integrated process record edition section 16 associates the manufacturing conditions of each process with the quality of the metal material manufactured under these manufacturing conditions for each of predetermined areas in the length direction of the metal material, and stores them in the record database 17. The following describes an example of processing by the integrated process record edition section 16.

For example, a case is considered in which a metal material (material) is manufactured through process 1, process 2, and process 3 as illustrated in FIG. 5. Processes 1 through 3 are rolling processes, for example, and the longitudinal length of the material increases with each process. As illustrated in FIG. 5, material A is divided into material A1 and material A2 when moving from process 1 to process 2, and material A1 is divided into material A11 and material A12 when moving from process 2 to process 3.

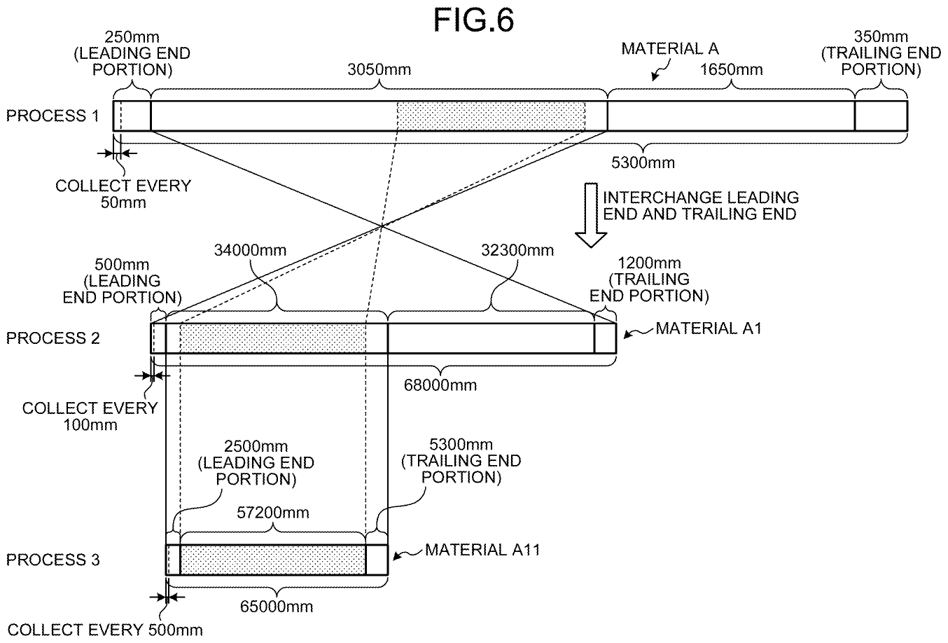

FIG. 6 illustrates an image of the materials in each process, focusing on part B in FIG. 5. For example, for material A in process 1, the manufacturing record collection section 11 collects record data for M1 items, for example, X₁¹ to X₁^{M1}, every 50 mm in an area of 5300 mm in length from the leading end to the trailing end. For material A, the cutting record collection section 15 collects record data indicating that the leading end portion from 0 mm (leading end) to 250 mm has been truncated, material A1 has been taken from 250 mm to 3300 mm, material A2 has been taken from 3300 mm to 4950 mm, and the trailing end portion from 4950 mm to 5300 mm (trailing end) has been truncated.

Then, for material A1 in process 2, the leading-trailing end interchange record collection section 13 collects the record data of “the leading and trailing end interchanged”. For material A1, the manufacturing record collection section 11 collects record data for M2 items, for example, X₂¹ to X₂^{M2}, every 100 mm in an area of 68000 mm in length from the leading end to the trailing end. For material A1, the cutting record collection section 15 collects record data indicating that the leading end portion from 0 mm (leading end) to 500 mm has been truncated, material A11 has been taken from 500 mm to 34500 mm, material A12 has been taken from 34500 mm to 66800 mm, and the trailing end portion from 66800 mm to 68000 mm (trailing end) has been truncated.

Then, for material A11 in process 3, the leading-trailing end interchange record collection section 13 collects the record data of “the leading and trailing ends not interchanged”. For material A11, the manufacturing record collection section 11 collects record data for M3 items, for example, X₃¹ to X₃^{M3}, every 500 mm in an area of 65000 mm in length from the leading end to the trailing end. For material A11, the cutting record collection section 15 collects record data indicating that the leading end portion from 0 mm (leading end) to 2500 mm has been truncated, material A11 has been taken from 2500 mm to 59700 mm, and the trailing end portion from 59700 mm to 65000 mm (trailing end) has been truncated.

In order to combine the record data of all processes, which are collected in detail in the longitudinal direction by sensors not illustrated in the drawings, in units of the length of the metal materials in the final process, while taking into account, for every metal material in each process, the record data on whether the leading end and the trailing end have been interchanged, whether the front face and the back face have been interchanged, and the cutting position and others, the integrated process record edition section 16 scales the material lengths in process 2 and process 1 to match the material length in process 3, the final process, as illustrated in FIG. 5 (refer to the dashed line in FIG. 5).

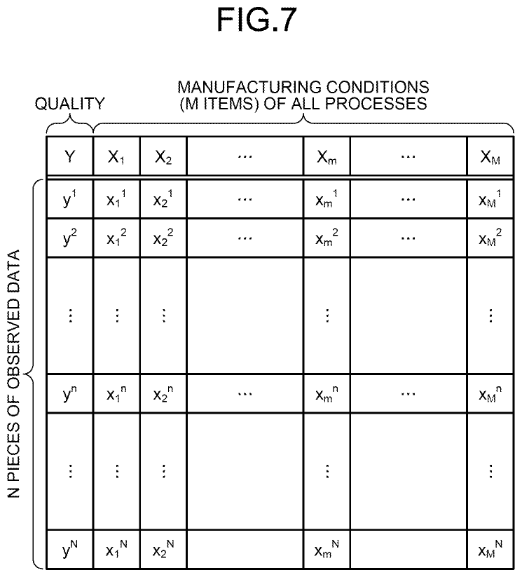

The integrated process record edition section 16 then identifies the position from which each metal material was taken, while taking into account the leading end portion and the trailing end portion truncated in each process, and, for the predetermined area of each metal material in the final process, associates the quality in the predetermined area with the manufacturing conditions of all processes in that predetermined area, and stores them in the record database 17. For example, in FIG. 6, the shaded area from which material A11 was taken in the final process, process 3, is identified back to material A1 in process 2 and material A in process 1. By repeating such processing for all metal materials, as illustrated in FIG. 7, record data on the manufacturing conditions (and quality) of metal materials in all processes are aligned and combined in units of the length of the metal materials. The following description returns back to FIG. 2 and continues.

The model generation section 18 generates a quality prediction model that predicts the quality of a metal material for each of predetermined areas based on the manufacturing condition for each of the predetermined areas in each process (step S4). Then, the prediction section 19 uses the quality prediction model generated by the model generation section 18 to predict the quality of a metal material manufactured under a certain manufacturing condition for each of predetermined areas (step S5).

According to the quality prediction model generation method, the quality prediction model, the quality prediction method, the quality prediction model generation device, and the quality prediction device according to the present embodiment as described above, by generating a quality prediction model that associates a manufacturing condition of each process with the quality of a metal material manufactured under the manufacturing condition for each of predetermined areas, the quality of a metal material with respect to a certain manufacturing condition can be predicted more accurately than it is conventionally.

In other words, the quality prediction model generation method, the quality prediction model, the quality prediction method, the quality prediction model generation device, and the quality prediction device according to the present embodiment combines the record data of the manufacturing condition (and quality) of all processes while aligning them in units of the length of the metal material at the output side of the final process, by taking into account, for each process, whether the leading end and the trailing end of the metal material have been interchanged, whether the front face and the back face of the metal material have been interchanged, and the cutting position and others. Thus, the record data of the manufacturing condition that are collected in detail in the longitudinal direction of the metal material by sensors, is effectively used to predict quality, so that the quality can be predicted with higher accuracy than it is conventionally.

When the quality prediction method according to the present embodiment is applied to a manufacturing method of a metal material, for example, the following processing is performed. First, a manufacturing condition confirmed during manufacturing of the metal material is fixed, and then the quality of the metal material manufactured under the fixed manufacturing condition is predicted for each of predetermined areas by the quality prediction method according to the present embodiment. Second, the manufacturing condition of a subsequent process is then changed based on the predicted results. The manufacturing condition is changed such that the quality of the manufactured metal material in every predetermined area included over the entire length of the metal material is within a predetermined control range. Thus, by applying the quality prediction method according to the present embodiment to a manufacturing method of a metal material, the final quality of the metal material can be predicted at a stage during manufacturing and a manufacturing condition can be changed accordingly, and thus the quality of the metal material to be manufactured is improved.

### EXAMPLES

Examples of the quality prediction method according to the present embodiment will be explained. In an example, the quality prediction method according to the present embodiment was applied to the prediction of tensile strength of a highly workable high-strength cold-rolled steel sheet, which is a type of cold-rolled thin steel sheet.

The objective variable (quality) of the quality prediction in this example is the tensile strength of a product (highly workable high-strength cold-rolled steel sheet), and the explanatory variables (manufacturing conditions) are the chemical composition of the metal material in the smelting process, the temperature of the metal material in the casting process, the temperature of the metal material in the heating process, the temperature of the metal material in the hot rolling process, the temperature of the metal material in the cooling process, the temperature of the metal material in the cold rolling process, the temperature of the metal material in the annealing process, and others.

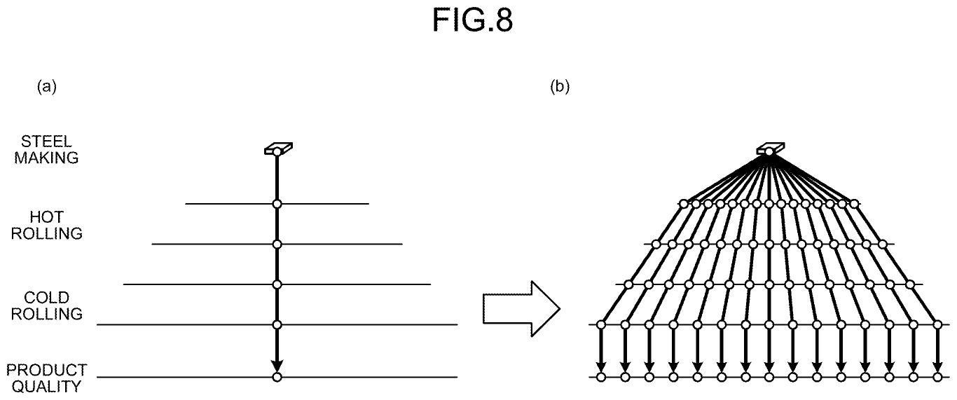

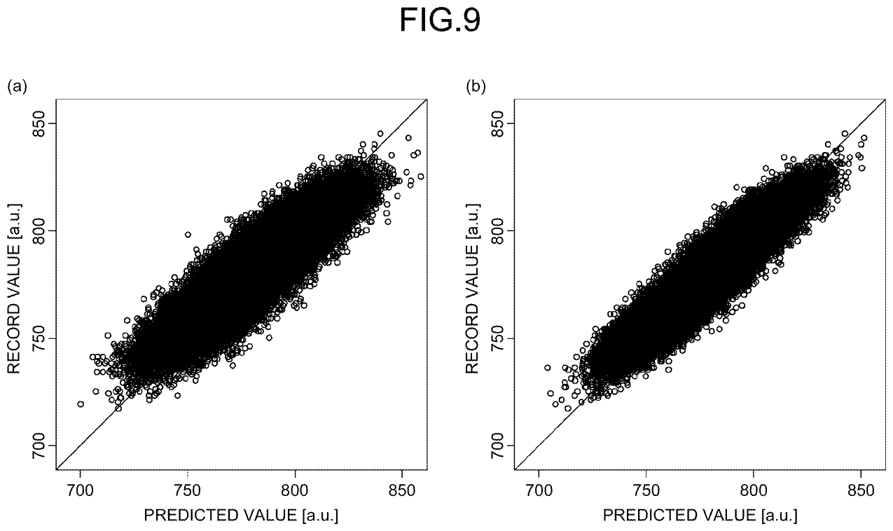

In this example, prediction results were compared between a case where prediction was made based on a conventional record database (refer to FIG. 8(a)) in which one representative value, such as an average value, for each manufacturing condition and each quality is stored for each product, and a case where prediction was made based on a quality prediction model generated from a record database (refer to FIG. 8(b)) of the quality prediction method according to the present embodiment. The number of samples in the record database was 40000, the number of explanatory variables was 45, and the prediction method used was local regression. As a record of the prediction, as illustrated in FIG. 9, the prediction error by the quality prediction method according to the present embodiment (refer to FIG. 9(b)) was confirmed that the root mean square error (RMSE) can be reduced by 23% compared to the prediction error by the conventional quality prediction method (refer to FIG. 9(a)).

The quality prediction method according to the present embodiment was also applied to the prediction of the front and back surface hardness of a thick steel sheet. The objective variable is the front and back surface hardness of a product, and the explanatory variables are the chemical composition in the smelting process, the front and back surface temperatures in the casting process, the front and back surface temperatures in the heating process, the front and back surface temperatures in the rolling process, the front and back surface temperatures in the cooling process, and others.

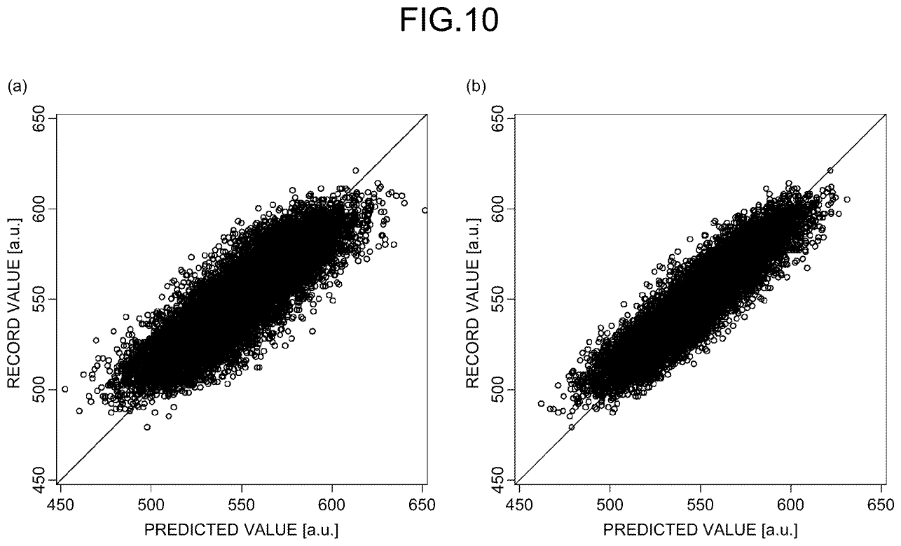

Prediction records were compared between a case where prediction was made based on a conventional record database (refer to FIG. 8(a)) in which one representative value, such as an average value, for each manufacturing condition and each quality is stored for each product, and a case where prediction was made based on a quality prediction model generated from a record database (refer to FIG. 8(b)) of the quality prediction method according to the present embodiment. The number of samples in the record database was 10000, the number of explanatory variables was 30, and the prediction method used was linear regression. As a record of the prediction, as illustrated in FIG. 10, the prediction error by the quality prediction method according to the present embodiment (refer to FIG. 10(b)) was confirmed that the RMSE can be reduced by 26% compared to the prediction error by the conventional quality prediction method (refer to FIG. 10(a)).

The quality prediction method according to the present embodiment was also applied to the prediction of front and back surface defects of a hot-dip galvanized steel sheet, which is a type of cold-rolled thin steel sheet. The objective variable was the presence or absence of the front and back surface defects on the product, and the explanatory variables are the chemical composition in the smelting process, the front and back surface temperatures, the meniscus flow rate, and the molten metal surface level in the casting process, the front and back surface temperatures in the heating process, the front and back surface temperatures in the hot rolling process, the front and back surface temperatures in the cooling process, the acid concentration and acid temperature in the pickling process, the front and back surface temperatures in the cold pressing process, the front and back surface temperatures in the annealing process, the amount of plating adhesion and the degree of alloying in the plating process, and others.

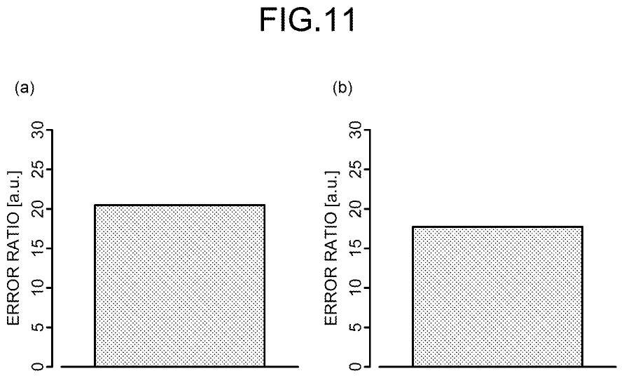

Prediction records were compared between a case where prediction was made based on a conventional record database (refer to FIG. 8(a)) in which one representative value, such as an average value, for each manufacturing condition and each quality is stored for each product, and a case where prediction was made based on a quality prediction model generated from a record database (refer to FIG. 8(b)) of the quality prediction method according to the present embodiment. The number of samples in the record database was 4,000, the number of explanatory variables was 250, and the prediction method used was decision trees. As a record of the prediction, as illustrated in FIG. 11, it was confirmed that the error ratio by the quality prediction method according to the present embodiment (refer to FIG. 11(b)) can be reduced by 14% compared to the error ratio by the conventional quality prediction method (refer to FIG. 11(a)).

The quality prediction method according to the present embodiment was also applied to the prediction of tensile strength of a high-strength cold-rolled steel sheet, which is a type of cold-rolled thin steel sheet, and a manufacturing condition of a subsequent process was changed based on the prediction records. The following describes an example of changing the post-annealing cooling temperature, which is a manufacturing condition for the final stage of the cold rolling process, at a stage in manufacturing where the record values of manufacturing conditions of the steel making process, the hot rolling process, and up to a stage previous to the final stage of the cold rolling process have been obtained.

On the basis of the record values of the manufacturing conditions of the steel making process, the hot rolling process, and up to the stage previous to the final stage of the cold rolling process, and a reference value of the post-annealing cooling temperature, which is a manufacturing condition for the final stage of the cold rolling process, the tensile strength values at positions in the total length of the product predicted using the quality prediction method according to the present embodiment are presented as follows:

ŷ¹,ŷ², . . . ŷ^{L}

When the cooling temperature after the annealing temperature changes by Au from the reference value, the amounts of change in tensile strength at the positions in the total length of the product predicted using the quality prediction method according to the present embodiment are presented as follows:

Δŷ¹(Δu),Δŷ²(Δu), . . . Δŷ^{L}(Δu)

On the basis of the above, the optimization problem expressed in the following equation (1) is solved:

$$Δ {u}^{⋆} = arg {min}_{Δ u} \vert Δ u \vert … subject to … {y}_{L L} \leq {\hat{y}}^{k} + Δ {\hat{y}}^{k} ( Δ u ) \leq {y}_{UL} , \\ ( k = {1}_{,} 2 , … , L ) \qquad ( 1 )$$

Here, in the above equation (1), y_{LL} and y_{UL} are the lower and upper control limits of tensile strength, respectively, and Δμ* is the optimal solution of this optimization problem. This optimization problem can be solved by a mathematical programming method such as the branch-and-bound method. By changing the cooling temperature after the annealing temperature by Δμ*, it is possible to obtain a cold-rolled steel sheet in which the tensile strength of the entire length does not fall outside the control range, that is, there is no quality defect over the entire length.

As described above, the record data stored in the record database of the quality prediction method according to the present embodiment can be combined in a manner that hardness or defect presence can be retroactively combined with detailed manufacturing conditions of all processes, while taking into account the record data on whether the leading end and the trailing end have been interchanged, whether the front face and the back face have been interchanged, and the cutting position and others in each predetermined area of the metal material in the final process. Then, based on the quality prediction model generated from the record database so constructed, a predicted value under a certain manufacturing condition is calculated, making it possible to predict the quality of a metal material with high accuracy.

The quality prediction model generation method, the quality prediction model, the quality prediction method, the metal material manufacturing method, the quality prediction model generation device, and the quality prediction device according to aspects of the present invention have been described in detail in the descriptions of embodiments and examples. The purpose of the present invention is, however, not limited to these descriptions, and should be broadly interpreted based on the descriptions in the scope of the claims. In addition, it goes without saying that various changes and modifications based on these descriptions are also included in aspects of the present invention.

For example, in the aforementioned embodiment, the integrated process record edition section 16 identifies a predetermined area by taking into account, for every metal material in each process, whether the leading end and the trailing end have been interchanged, whether the front face and the back face have been interchanged, and the cutting position; however, not all of the processes of interchanging the leading and trailing ends of the metal material, interchanging the front and back faces of the metal material, and cutting the metal material are necessarily included in some cases. Therefore, the integrated process record edition section 16 may identify the predetermined area by taking into account at least one or more of the record data on: whether the leading end and the trailing end of the metal material have been interchanged; whether the front face and the back face of the metal material have been interchanged; and the cutting position of the metal material.

### REFERENCE SIGNS LIST

1 quality prediction device
11 manufacturing record collection section
12 manufacturing record edition section
13 leading-trailing end interchange record collection section
14 front-back face interchange record collection section
15 cutting record collection section
16 integrated process record edition section
17 record database
18 model generation section
19 prediction section

1. A quality prediction model generation method for a metal material manufactured through one or more processes, the method comprising:
a first collection step of collecting a manufacturing condition of each of the processes for each of predetermined areas of the metal material;
a second collection step of evaluating and collecting quality of the metal material manufactured through each process for each of the predetermined areas;
a storage step of storing the manufacturing condition of each process and the quality of the metal material manufactured under the manufacturing condition in association with each other for each of the predetermined areas; and
a model generation step of generating a quality prediction model that predicts quality of the metal material for each of the predetermined areas based on the stored manufacturing condition for each of the predetermined areas in each process.

2. The quality prediction model generation method according to claim 1, wherein each of the predetermined areas is determined based on a travel distance of the metal material in a conveyance direction in each process.

3. The quality prediction model generation method according to claim 1, comprising, before the storage step, a third collection step of collecting at least one or more of whether a leading end and a trailing end of the metal material have been interchanged in each process, whether a front face and a back face of the metal material have been interchanged in each process, and a cutting position of the metal material in each process, wherein
the storage step identifies the predetermined areas by taking into account, on the metal material in each process, at least one or more of whether the leading end and the trailing end have been interchanged, whether the front face and the back face have been interchanged, and the cutting position, and stores the manufacturing condition of each process and the quality of the metal material produced under the manufacturing condition in association with each other for each of the predetermined areas.

4. The quality prediction model generation method according to claim 1, wherein
in a case where a shape of the metal material is deformed by going through each process,
the storage step identifies the predetermined area by evaluating a volume of the metal material from the leading end, and stores the manufacturing condition of each process and the quality of the metal material manufactured under the manufacturing condition in association with each other for each of the predetermined areas.

5. The quality prediction model generation method according to claim 1, wherein the model generation step generates the quality prediction model using machine learning including linear regression, local regression, principal component regression, PLS regression, a neural network, a regression tree, a random forest, and XGBoost.

6. A quality prediction model generated by the quality prediction model generation method according to claim 1.

7. A quality prediction method comprising
predicting quality of a metal material manufactured under a certain manufacturing condition for each of the predetermined areas using a quality prediction model generated by the quality prediction model generation method according to claim 1.

8. A metal material manufacturing method comprising:
fixing a manufacturing condition confirmed during manufacturing;
predicting quality of a metal material manufactured under the fixed manufacturing condition for each of predetermined areas by the quality prediction method according to claim 7; and
changing the manufacturing condition of a subsequent process based on a predicted result.

9. The metal material manufacturing method according to claim 8, wherein the changing the manufacturing condition is performed such that the quality of the manufactured material in every predetermined area included over an entire length of the metal material is within a predetermined control range.

10. A quality prediction model generation device for a metal material manufactured through one or more processes, the device comprising:
a collecting unit configured to collect a manufacturing condition of each of the processes for each of predetermined areas of the metal material;
an evaluating unit configured to evaluate and collect quality of the metal material manufactured through each process for each of the predetermined areas;
a storing unit configured to store the manufacturing condition of each process and the quality of the metal material manufactured under the manufacturing condition in association with each other for each of the predetermined areas; and
a generating unit configured to generate a quality prediction model that predicts quality of the metal material for each of the predetermined areas based on the stored manufacturing condition for each of the predetermined areas in each process.

11. A quality prediction device configured to predict quality of a metal material manufactured under a certain manufacturing condition for each of the predetermined areas using a quality prediction model generated by the quality prediction model generation device according to claim 10.
